(19) United States
(12) Patent Application Publication
(10) Pub. No.: US 2018/0235701 A1
(43) Pub. Date: Aug. 23, 2018

(54) SYSTEMS AND METHODS FOR INTERVENTION GUIDANCE USING PRE-OPERATIVE PLANNING WITH ULTRASOUND

(71) Applicant: General Electric Company, Schenectady, NY (US)

(72) Inventors: Olivier Gerard, Oslo (NO); Stian Langeland, Vollen (NO); Eigil Samset, Oppegard (NO); Maxime Cazalas, Paris (FR)

(21) Appl. No.: 15/438,386

(22) Filed: Feb. 21, 2017

(51) Int. Cl. A61B 34/10; A61B 8/08; A61B 8/00; A61B 6/00; A61B 6/04; A61B 5/055; A61B 6/03

(52) U.S. Cl. CPC A61B 34/10; A61B 8/483; A61B 8/463; A61B 6/463; A61B 6/0407; A61B 6/487; A61B 8/5246; A61B 6/032; A61B 6/4435; A61B 6/4266; A61B 6/5205; A61B 8/467; A61B 8/5261; A61B 5/055

(57) ABSTRACT

Methods and systems are provided for multi-modality imaging. In one embodiment, a method comprises: receiving planning annotations of a pre-operative three-dimensional (3D) image of a subject; during an ultrasound scan of the subject, registering an ultrasound image with the 3D image; overlaying the planning annotations on the ultrasound image; and displaying the ultrasound image with the overlaid planning annotations. In this way, pre-operative planning by a physician can be readily used during intervention.

### FIELD

Embodiments of the subject matter disclosed herein relate to multi-modality imaging, and more particularly, to interventional cardiology.

### BACKGROUND

Presently available medical imaging technologies such as ultrasound imaging, computed tomography (CT) imaging, and x-ray fluoroscopic imaging are known to be helpful not only for non-invasive diagnostic purposes, but also for providing assistance during surgery. For example, during cardiac interventions, ultrasound imaging is often utilized for guidance and monitoring of the procedure. X-ray angiography may also be used in conjunction with ultrasound during cardiac interventions to provide additional guidance. Ultrasound images include more anatomical information of cardiac structures than x-ray images which do not effectively depict soft structures, while x-ray images more effectively depict catheters and other surgical instruments than ultrasound images.

### BRIEF DESCRIPTION

In one embodiment, a method comprises: receiving planning annotations of a three-dimensional (3D) image of a subject; during an ultrasound scan of the subject, registering an ultrasound image with the 3D image; overlaying the planning annotations on the ultrasound image; and displaying the ultrasound image with the overlaid planning annotations. In this way, pre-operative planning by a physician can be readily used during intervention.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 illustrates an ultrasound system interconnected with an x-ray fluoroscopic system formed in accordance with an embodiment;

FIG. 2 shows a block diagram illustrating an example computed tomography (CT) imaging system in accordance with an embodiment;

FIG. 3 shows a block diagram illustrating an example magnetic resonance imaging (MRI) system in accordance with an embodiment; and

FIG. 4 shows a high-level flow chart illustrating an example method for displaying pre-operative planning information during an intervention according to an embodiment.

### DETAILED DESCRIPTION

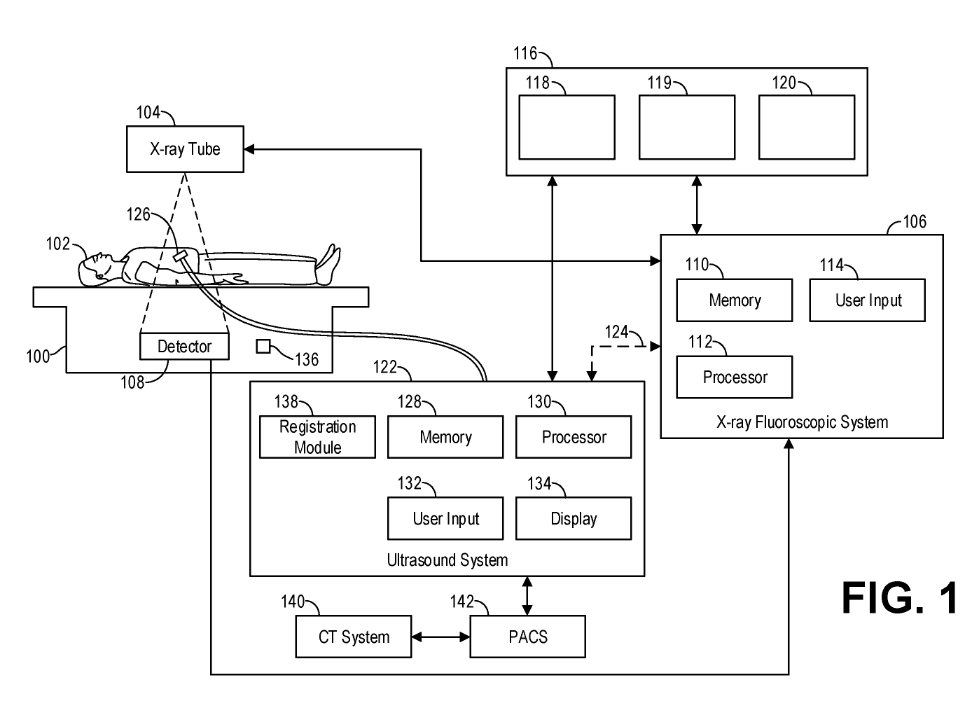

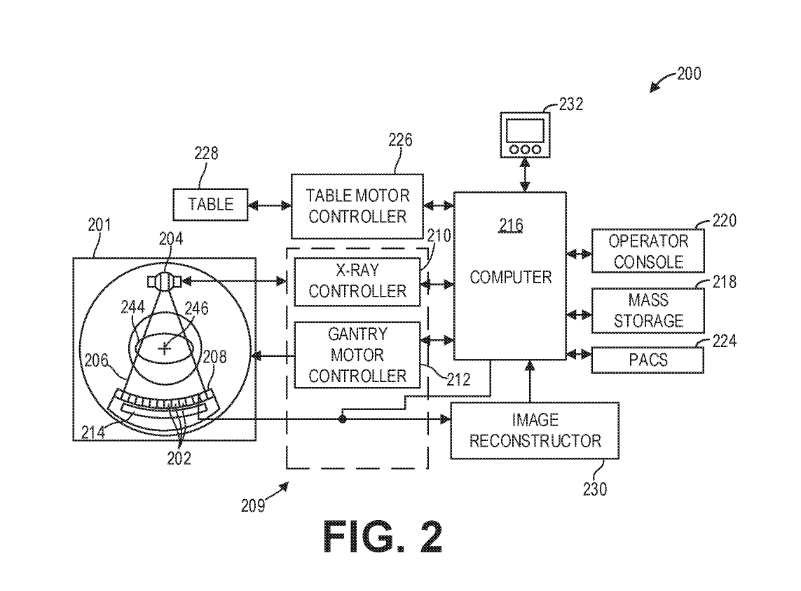

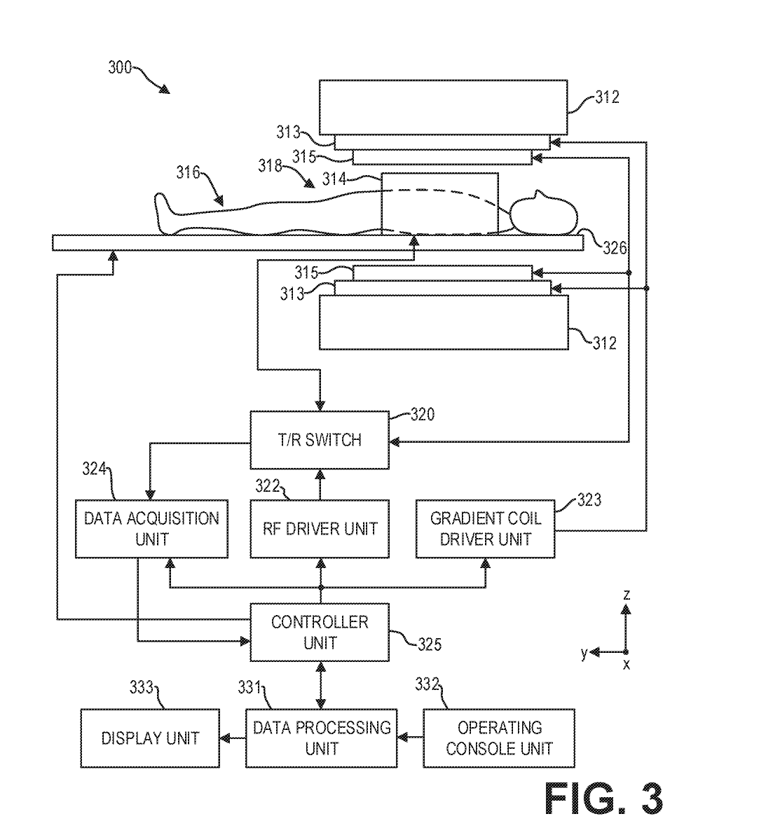

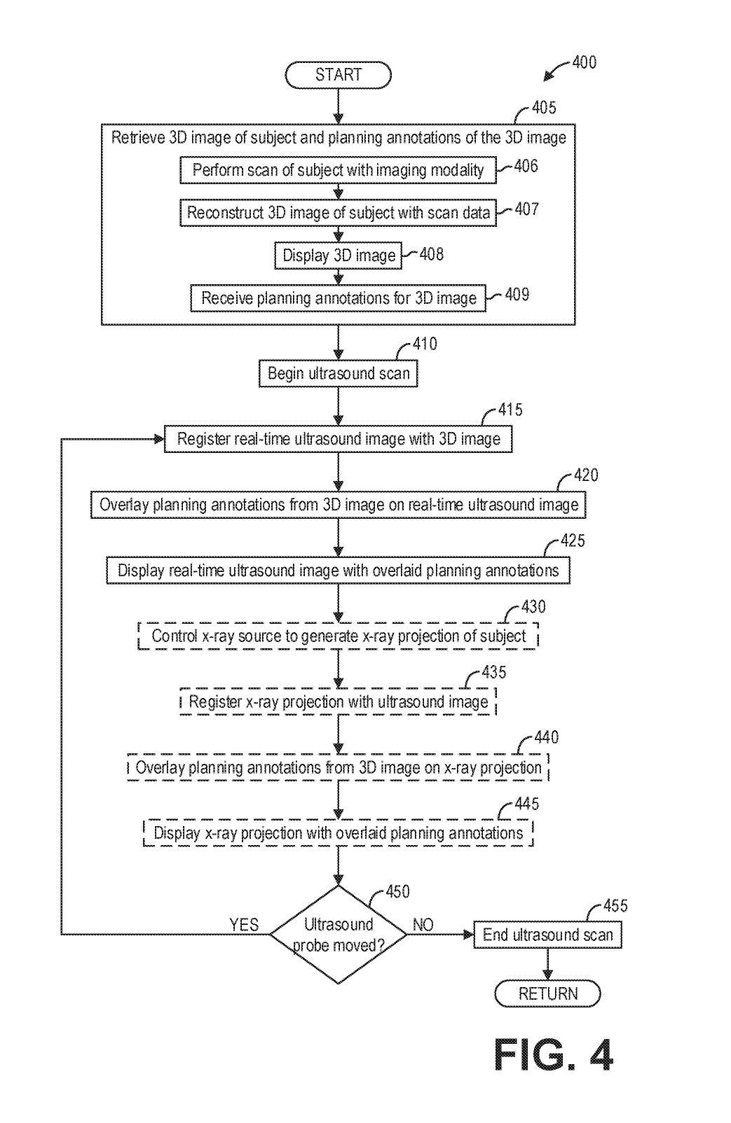

The following description relates to various embodiments of multi-modality imaging. In particular, systems and methods are provided for intervention guidance using pre-operative planning with ultrasound. A multi-modality imaging system for interventional procedures, such as the system depicted in FIG. 1, may include multiple imaging modalities, including but not limited to computed tomography (CT), magnetic resonance imaging (MM), ultrasound, and x-ray fluoroscopy. Pre-operative diagnostic three-dimensional (3D) images may be acquired with a 3D imaging modality, such as the CT imaging system depicted in FIG. 2 or the MM system depicted in FIG. 3, respectively. Such pre-operative 3D images may be used to plan an intervention. A method for providing interventional guidance, such as the method depicted in FIG. 4, may overlay annotations to the pre-operative 3D images made by a physician or another user on live ultrasound images and/or x-ray projection images, such that the planning annotations may be utilized in real-time during an intervention.

FIG. 1 illustrates a multi-modality imaging system 10 in accordance with an embodiment of the present invention. Multi-modality imaging system 10 may include an x-ray fluoroscopic system 106, an ultrasound system 122, and a 3D imaging modality 140.

A table 100 or bed is provided for supporting a subject 102. An x-ray tube 104 or other generator is connected to an x-ray fluoroscopic system 106. As shown, the x-ray tube 104 is positioned above the subject 102, but it should be understood that the x-ray tube 104 may be moved to other positions with respect to the subject 102. A detector 108 is positioned opposite the x-ray tube 104 with the subject 102 there-between. The detector 108 may be any known detector capable of detecting x-ray radiation.

The x-ray fluoroscopic system 106 has at least a memory 110, a processor 112, and at least one user input 114, such as a keyboard, trackball, pointer, touch panel, and the like. To acquire an x-ray image, the x-ray fluoroscopic system 106 causes the x-ray tube 104 to generate x-rays and the detector 108 detects an image. Fluoroscopy may be accomplished by activating the x-ray tube 104 continuously or at predetermined intervals while the detector 108 detects corresponding images. Detected image(s) may be displayed on a display 116 that may be configured to display a single image or more than one image at the same time.

In some examples, the ultrasound system 122 communicates with the x-ray fluoroscopic system 106 via an optional connection 124. The connection 124 may be a wired or wireless connection. The ultrasound system 122 may transmit or convey ultrasound imaging data to the x-ray fluoroscopic system 106. The communication between the systems 106 and 122 may be one-way or two-way, allowing image data, commands, and information to be transmitted between the two systems 106 and 122. The ultrasound system 122 may be a stand-alone system that may be moved from room to room, such as a cart-based system, hand-carried system, or other portable system.

An operator (not shown) may position an ultrasound probe 126 on the subject 102 to image an area of interest within the subject 102. The ultrasound system 122 has at least a memory 128, a processor 130, and a user input 132. Optionally, if the ultrasound system 122 is a stand-alone system, a display 134 may be provided. By way of example, images acquired using the x-ray fluoroscopic system 106 may be displayed as a first image 118 and images acquired using the ultrasound system 122 may be displayed as a second image 120 on the display 116, forming a dual display configuration. In another embodiment, two side-by-side monitors (not shown) may be used. The images acquired by both the x-ray fluoroscopic system 106 and the ultrasound system 122 may be acquired in known manners.

In one embodiment, the ultrasound system 122 may be a 3D-capable miniaturized ultrasound system that is connected to the x-ray fluoroscopic system 106 via the connection 124. As used herein, “miniaturized” means that the ultrasound system 122 is configured to be carried in a person's hand, pocket, briefcase-sized case, or backpack. For example, the ultrasound system 122 may be a hand-carried device having a size of a typical laptop computer, for instance, having dimensions of approximately 2.5 inches in depth, approximately 14 inches in width, and approximately 12 inches in height. The ultrasound system 122 may weigh approximately ten pounds, and thus is easily portable by the operator. An integrated display, such as the display 134, may be configured to display an ultrasound image as well as an x-ray image acquired by the x-ray fluoroscopic system 106.

As another example, the ultrasound system 122 may be a 3D-capable pocket-sized ultrasound system. By way of example, the pocket-sized ultrasound system may be approximately 2 inches wide, approximately 4 inches in length, and approximately 0.5 inches in depth, and weigh less than 3 ounces. The pocket-sized ultrasound system may include a display (e.g., the display 134), a user interface (e.g., user input 132), and an input/output (I/O) port for connection to the probe 126. It should be noted that the various embodiments may be implemented in connection with a miniaturized or pocket-sized ultrasound system having different dimensions, weights, and power consumption.

In another embodiment, the ultrasound system 122 may be a console-based ultrasound imaging system provided on a movable base. The console-based ultrasound imaging system may also be referred to as a cart-based system. An integrated display (e.g., the display 134) may be used to the display the ultrasound image alone or simultaneously with the x-ray image as discussed herein.

In yet another embodiment, the x-ray fluoroscopic system 106 and the ultrasound system 122 may be integrated together and may share at least some processing, user input, and memory functions. For example, a probe port 136 may be provided on the table 100 or other apparatus near the subject 102. The probe 126 may thus be connected to the probe port 136.

In some examples, a pre-operative 3D image 119 of the patient 102 may be acquired with the 3D imaging modality 140. The 3D imaging modality 140 may comprise, as illustrative and non-limiting examples, a computed tomography (CT) imaging system or a magnetic resonance imaging (MRI) system. For example, the 3D imaging modality 140 may comprise a CT imaging system configured to generate three-dimensional images of a subject. As described further below with regard to FIG. 2, the CT imaging system may include an x-ray radiation source configured to project a beam of x-ray radiation towards a detector array positioned on the opposite side of a gantry to which the radiation source is mounted. The CT system may further include a computing device that controls system operations such as data acquisition and/or processing. The computing device may be configured to reconstruct three-dimensional images from projection data acquired via the detector array, and such images may be stored locally or remotely in a picture archiving and communications system (PACS) such as PACS 142.

As another example, the 3D imaging modality 140 may comprise an MM system that transmits electromagnetic pulse signals to the subject placed in an imaging space with a magnetostatic field formed to perform a scan for obtaining magnetic resonance signals from the subject to reconstruct a three-dimensional image of the subject based on the magnetic resonance signals thus obtained by the scan. As described further herein below with regard to FIG. 3, the MM system may include a magnetostatic field magnet, a gradient coil, a radiofrequency (RF) coil, a computing device, and so on as known in the art.

The 3D imaging modality 140 may include or may be coupled to a picture archiving and communications system (PACS) 142. As depicted, the ultrasound system 122 may also be coupled to the PACS 142. As described further herein with regard to FIG. 3, the ultrasound system 122 may include a registration module 138 configured to register the ultrasound image 118 and the 3D image 119 retrieved from the PACS 142 with respect to each other. As described further herein with regard to FIG. 2, after aligning or registering ultrasound image 118 to the 3D image 119, planning annotations for the 3D image 119 may be overlaid on the ultrasound image 118.

In an exemplary implementation, the PACS 142 is further coupled to a remote system such as a radiology department information system, hospital information system, and/or to an internal or external network (not shown) to allow operators at different locations to supply commands and parameters and/or gain access to the image data.

FIG. 2 illustrates an exemplary computed tomography (CT) imaging system 200 configured to allow fast and iterative image reconstruction. Particularly, the CT system 200 is configured to image a subject such as a patient, an inanimate object, one or more manufactured parts, and/or foreign objects such as dental implants, stents, and/or contrast agents present within the body. In one embodiment, the CT system 200 includes a gantry 201, which in turn, may further include at least one x-ray radiation source 204 configured to project a beam of x-ray radiation 206 for use in imaging the patient. Specifically, the radiation source 204 is configured to project the x-rays 206 towards a detector array 208 positioned on the opposite side of the gantry 201. Although FIG. 2 depicts only a single radiation source 204, in certain embodiments, multiple radiation sources may be employed to project a plurality of x-rays 206 for acquiring projection data corresponding to the patient at different energy levels.

In one embodiment, the system 200 includes the detector array 208. The detector array 208 further includes a plurality of detector elements 202 that together sense the x-ray beams 206 that pass through a subject 244 such as a patient to acquire corresponding projection data. Accordingly, in one embodiment, the detector array 208 is fabricated in a multi-slice configuration including the plurality of rows of cells or detector elements 202. In such a configuration, one or more additional rows of the detector elements 202 are arranged in a parallel configuration for acquiring the projection data.

In certain embodiments, the system 200 is configured to traverse different angular positions around the subject 244 for acquiring desired projection data. Accordingly, the gantry 201 and the components mounted thereon may be configured to rotate about a center of rotation 246 for acquiring the projection data, for example, at different energy levels. Alternatively, in embodiments where a projection angle relative to the subject 244 varies as a function of time, the mounted components may be configured to move along a general curve rather than along a segment of a circle.

In one embodiment, the system 200 includes a control mechanism 209 to control movement of the components such as rotation of the gantry 201 and the operation of the x-ray radiation source 204. In certain embodiments, the control mechanism 209 further includes an x-ray controller 210 configured to provide power and timing signals to the radiation source 204. Additionally, the control mechanism 209 includes a gantry motor controller 212 configured to control a rotational speed and/or position of the gantry 201 based on imaging requirements.

In certain embodiments, the control mechanism 209 further includes a data acquisition system (DAS) 214 configured to sample analog data received from the detector elements 202 and convert the analog data to digital signals for subsequent processing. The data sampled and digitized by the DAS 214 is transmitted to a computing device 216. In one example, the computing device 216 stores the data in a storage device 218. The storage device 218, for example, may include a hard disk drive, a floppy disk drive, a compact disk-read/write (CD-R/W) drive, a Digital Versatile Disc (DVD) drive, a flash drive, and/or a solid-state storage device.

Additionally, the computing device 216 provides commands and parameters to one or more of the DAS 214, the x-ray controller 210, and the gantry motor controller 212 for controlling system operations such as data acquisition and/or processing. In certain embodiments, the computing device 216 controls system operations based on operator input. The computing device 216 receives the operator input, for example, including commands and/or scanning parameters via an operator console 220 operatively coupled to the computing device 216. The operator console 220 may include a keyboard (not shown) and/or a touchscreen to allow the operator to specify the commands and/or scanning parameters.

Although FIG. 2 illustrates only one operator console 220, more than one operator console may be coupled to the system 200, for example, for inputting or outputting system parameters, requesting examinations, and/or viewing images. Further, in certain embodiments, the system 200 may be coupled to multiple displays, printers, workstations, and/or similar devices located either locally or remotely, for example, within an institution or hospital, or in an entirely different location via one or more configurable wired and/or wireless networks such as the Internet and/or virtual private networks.

In one embodiment, for example, the system 200 either includes, or is coupled to a picture archiving and communications system (PACS) 224. In an exemplary implementation, the PACS 224 is further coupled to a remote system such as a radiology department information system, hospital information system, and/or to an internal or external network (not shown) to allow operators at different locations to supply commands and parameters and/or gain access to the image data.

The computing device 216 uses the operator-supplied and/or system-define commands and parameters to operate a table motor controller 226, which in turn, may control a motorized table 228. Particularly, the table motor controller 226 moves the table 228 for appropriately positioning the subject 244 in the gantry 201 for acquiring projection data corresponding to the target volume of the subject 244.

As previously noted, the DAS 214 samples and digitizes the projection data acquired by the detector elements 202. Subsequently, an image reconstructor 230 uses the sampled and digitized x-ray data to perform high-speed reconstruction. In certain embodiments, the image reconstructor 230 is configured to reconstruct images of a target volume of the patient using an iterative or analytic image reconstruction method. For example, the image reconstructor 230 may use an analytic image reconstruction approach such as filtered backprojection (FBP) to reconstruct images of a target volume of the patient. As another example, the image reconstructor 230 may use an iterative image reconstruction approach such as advanced statistical iterative reconstruction (ASIR), conjugate gradient (CG), maximum likelihood expectation maximization (MLEM), model-based iterative reconstruction (MBIR), and so on to reconstruct images of a target volume of the patient.

Although FIG. 2 illustrates the image reconstructor 230 as a separate entity, in certain embodiments, the image reconstructor 230 may form part of the computing device 216. Alternatively, the image reconstructor 230 may be absent from the system 200 and instead the computing device 216 may perform one or more functions of the image reconstructor 230. Moreover, the image reconstructor 230 may be located locally or remotely, and may be operatively connected to the system 200 using a wired or wireless network. Particularly, one exemplary embodiment may use computing resources in a “cloud” network cluster for the image reconstructor 230.

In one embodiment, the image reconstructor 230 stores the reconstructed images in the storage device 218. Alternatively, the image reconstructor 230 transmits the reconstructed images to the computing device 216 for generating useful patient information for diagnosis and evaluation. In certain embodiments, the computing device 216 transmits the reconstructed images and/or the patient information to a display 232 communicatively coupled to the computing device 216 and/or the image reconstructor 230.

The various methods and processes described further herein may be stored as executable instructions in non-transitory memory on a computing device in system 200. In one embodiment, image reconstructor 230 may include such instructions in non-transitory memory, and may apply the methods described herein to reconstruct an image from scan data. In another embodiment, computing device 216 may include the instructions in non-transitory memory, and may apply the methods described herein, at least in part, to a reconstructed image after receiving the reconstructed image from image reconstructor 230. In yet another embodiment, the methods and processes described herein may be distributed across image reconstructor 230 and computing device 216.

In one embodiment, the display 232 allows the operator to evaluate the imaged anatomy. The display 232 may also allow the operator to select a volume of interest (VOI) and/or request patient information, for example, via graphical user interface (GUI) for a subsequent scan or processing.

As another example of a 3D imaging modality that may be utilized to acquire pre-operative 3D image(s) of a subject, FIG. 3 illustrates a magnetic resonance imaging (MM) apparatus 300 that includes a magnetostatic field magnet unit 312, a gradient coil unit 313, an RF coil unit 314, an RF body coil unit 315, a transmit/receive (T/R) switch 320, an RF port interface 321, an RF driver unit 322, a gradient coil driver unit 323, a data acquisition unit 324, a controller unit 325, a patient bed 326, a data processing unit 331, an operating console unit 332, and a display unit 333. The MM apparatus 300 transmits electromagnetic pulse signals to a subject 316 placed in an imaging space 318 with a magnetostatic field formed to perform a scan for obtaining magnetic resonance signals from the subject 316 to reconstruct an image of the slice of the subject 316 based on the magnetic resonance signals thus obtained by the scan.

The magnetostatic field magnet unit 312 includes, for example, typically an annular superconducting magnet, which is mounted within a toroidal vacuum vessel. The magnet defines a cylindrical space surrounding the subject 316, and generates a constant primary magnetostatic field along the Z direction of the cylinder space.

The MRI apparatus 310 also includes a gradient coil unit 313 that forms a gradient magnetic field in the imaging space 318 so as to provide the magnetic resonance signals received by the RF coil unit 314 with three-dimensional positional information. The gradient coil unit 313 includes three gradient coil systems, each of which generates a gradient magnetic field which inclines into one of three spatial axes perpendicular to each other, and generates a gradient field in each of frequency encoding direction, phase encoding direction, and slice selection direction in accordance with the imaging condition. More specifically, the gradient coil unit 313 applies a gradient field in the slice selection direction of the subject 316, to select the slice; and the RF coil unit 314 transmits an RF pulse to a selected slice of the subject 316 and excites it. The gradient coil unit 313 also applies a gradient field in the phase encoding direction of the subject 316 to phase encode the magnetic resonance signals from the slice excited by the RF pulse. The gradient coil unit 313 then applies a gradient field in the frequency encoding direction of the subject 316 to frequency encode the magnetic resonance signals from the slice excited by the RF pulse.

The RF coil unit 314 is disposed, for example, to enclose the region to be imaged of the subject 316. In the static magnetic field space or imaging space 318 where a static magnetic field is formed by the magnetostatic field magnet unit 312, the RF coil unit 314 transmits, based on a control signal from the controller unit 325, an RF pulse that is an electromagnet wave to the subject 316 and thereby generates a high-frequency magnetic field. This excites a spin of protons in the slice to be imaged of the subject 316. The RF coil unit 314 receives, as a magnetic resonance signal, the electromagnetic wave generated when the proton spin thus excited in the slice to be imaged of the subject 316 returns into alignment with the initial magnetization vector. The RF coil unit 314 may transmit and receive an RF pulse using the same RF coil.

The RF body coil unit 315 is disposed, for example, to enclose the imaging space 318, and produces RF magnetic field pulses orthogonal to the main magnetic field produced by the magnetostatic field magnet unit 312 within the imaging space 318 to excite the nuclei. In contrast to the RF coil unit 314, which may be easily disconnected from the MR apparatus 300 and replaced with another RF coil unit, the RF body coil unit 315 is fixedly attached and connected to the MR apparatus 300. Furthermore, whereas local coils such as those comprising the RF coil unit 314 can transmit to or receive signals from only a localized region of the subject 316, the RF body coil unit 315 generally has a larger coverage area and can be used to transmit or receive signals to the whole body of the subject 316. Using receive-only local coils and transmit body coils provides a uniform RF excitation and good image uniformity at the expense of high RF power deposited in the subject. For a transmit-receive local coil, the local coil provides the RF excitation to the region of interest and receives the MR signal, thereby decreasing the RF power deposited in the subject. It should be appreciated that the particular use of the RF coil unit 14 and/or the RF body coil unit 315 depends on the imaging application.

The T/R switch 320 can selectively electrically connect the RF body coil unit 315 to the data acquisition unit 324 when operating in receive mode, and to the RF driver unit 322 when operating in transmit mode. Similarly, the T/R switch 320 can selectively electrically connect the RF coil unit 314 to the data acquisition unit 324 when the RF coil unit 314 operates in receive mode, and to the RF driver unit 322 when operating in transmit mode. When the RF coil unit 314 and the RF body coil unit 315 are both used in a single scan, for example if the RF coil unit 314 is configured to receive MR signals and the RF body coil unit 315 is configured to transmit RF signals, then the T/R switch 320 may direct control signals from the RF driver unit 322 to the RF body coil unit 315 while directing received MR signals from the RF coil unit 314 to the data acquisition unit 324. The coils of the RF body coil unit 315 may be configured to operate in a transmit-only mode, a receive-only mode, or a transmit-receive mode. The coils of the local RF coil unit 314 may be configured to operate in a transmit-receive mode or a receive-only mode.

The RF driver unit 322 includes a gate modulator (not shown), an RF power amplifier (not shown), and an RF oscillator (not shown) that are used to drive the RF coil unit 314 and form a high-frequency magnetic field in the imaging space 318. The RF driver unit 322 modulates, based on a control signal from the controller unit 325 and using the gate modulator, the RF signal received from the RF oscillator into a signal of predetermined timing having a predetermined envelope. The RF signal modulated by the gate modulator is amplified by the RF power amplifier and then output to the RF coil unit 314.

The gradient coil driver unit 323 drives the gradient coil unit 313 based on a control signal from the controller unit 325 and thereby generates a gradient magnetic field in the imaging space 318. The gradient coil driver unit 323 includes three systems of driver circuits (not shown) corresponding to the three gradient coil systems included in the gradient coil unit 313.

The data acquisition unit 324 includes a preamplifier (not shown), a phase detector (not shown), and an analog/digital converter (not shown) used to acquire the magnetic resonance signals received by the RF coil unit 314. In the data acquisition unit 324, the phase detector phase detects, using the output from the RF oscillator of the RF driver unit 322 as a reference signal, the magnetic resonance signals received from the RF coil unit 314 and amplified by the preamplifier, and outputs the phase-detected analog magnetic resonance signals to the analog/digital converter for conversion into digital signals. The digital signals thus obtained are output to the data processing unit 331.

The MRI apparatus 300 includes a table 326 for placing the subject 316 thereon. The subject 316 may be moved inside and outside the imaging space 318 by moving the table 326 based on control signals from the controller unit 325.

The controller unit 325 includes a computer and a recording medium on which a program to be executed by the computer is recorded. The program when executed by the computer causes various parts of the apparatus to carry out operations corresponding to pre-determined scanning. The recording medium may comprise, for example, a ROM, flexible disk, hard disk, optical disk, magneto-optical disk, CD-ROM, or non-volatile memory card. The controller unit 325 is connected to the operating console unit 332 and processes the operation signals input to the operating console unit 332 and furthermore controls the table 326, RF driver unit 322, gradient coil driver unit 323, and data acquisition unit 324 by outputting control signals to them. The controller unit 325 also controls, to obtain a desired image, the data processing unit 331 and the display unit 333 based on operation signals received from the operating console unit 332.

The operating console unit 332 includes user input devices such as a keyboard and a mouse. The operating console unit 332 is used by an operator, for example, to input such data as an imaging protocol and to set a region where an imaging sequence is to be executed. The data about the imaging protocol and the imaging sequence execution region are output to the controller unit 325.

The data processing unit 331 includes a computer and a recording medium on which a program to be executed by the computer to perform predetermined data processing is recorded. The data processing unit 331 is connected to the controller unit 325 and performs data processing based on control signals received from the controller unit 325. The data processing unit 331 is also connected to the data acquisition unit 324 and generates spectrum data by applying various image processing operations to the magnetic resonance signals output from the data acquisition unit 324.

The display unit 333 includes a display device and displays an image on the display screen of the display device based on control signals received from the controller unit 325. The display unit 333 displays, for example, an image regarding an input item about which the operator inputs operation data from the operating console unit 332. The display unit 333 also displays a slice image of the subject 316 generated by the data processing unit 331.

It should be appreciated that although a CT system 200 and an MRI system 300 are depicted in FIGS. 2 and 3, respectively, such imaging modalities are illustrative and non-limiting, and any suitable 3D imaging modality may be utilized to acquire a pre-operative 3D image and provide interventional planning guidance or annotations.

FIG. 4 shows a high-level flow chart illustrating an example method 400 for interventional guidance using pre-operative planning for ultrasound imaging. In particular, method 400 relates to importing planning information provided using a pre-operative 3D image into a real-time ultrasound image and/or an x-ray projection image. Method 400 is described with regard to the systems and components described hereinabove with regard to FIGS. 1-3, though it should be understood that the method may be implemented with other systems and components without departing from the scope of the present disclosure. Method 400 may be stored as executable instructions in non-transitory memory, such as memory 128 of the ultrasound system 122, and executed by a processor, such as processor 130.

Method 400 begins at 405. At 405, method 400 retrieves a 3D image of a subject and planning annotations of the 3D image. For example, the 3D image and the planning annotations may be retrieved from a PACS such as PACS 142. As an illustrative and non-limiting example, at 406, method 400 may perform a scan of the subject, for example, using a 3D imaging modality 140. The 3D imaging modality may comprise any suitable imaging modality, such as the CT imaging system 200 depicted in FIG. 2 or the MRI system 300 depicted in FIG. 3. At 407, method 400 may reconstruct a 3D image of the subject using data acquired during the scan. At 408, method 400 displays the 3D image via a display device, such as display device 116. An operator may view the 3D image and prepare planning annotations using, for example, an operator console or another suitable user input device.

At 409, method 400 receives planning annotations for the 3D image. These planning annotations may comprise indications and delineations of specific anatomical features, spatial measurements for correct selection of intervention devices, simulation of device positioning, and so on. For example, if screws are to be used to fix a device to an anatomical structure, a user may use the three-dimensional image data to plan the position and orientation of each screw.

Thus the 3D image(s) and the planning annotations may be imported from the PACS into the ultrasound system. The 3D image and the planning annotations may be retrieved as two separate data entities or as a joint object (i.e., the planning annotations may be stored in the same file as the image).

It should be appreciated that 406, 407, 408, and 409 may be carried out by the 3D imaging modality during a pre-operative scanning session, and therefore may be implemented as executable instructions in non-transitory memory of the 3D imaging modality (e.g., of the computer 216 or the data processing unit 331, as non-limiting examples).

After importing the 3D image of the subject and the planning annotations, method 400 continues to 410. At 410, method 400 begins an ultrasound scan of the subject, for example with the ultrasound system 122. At 415, method 400 registers the real-time, three-dimensional ultrasound image with the 3D image retrieved at 405, for example via the registration module 138. The registration between the 3D image and the ultrasound image may be performed with a single echo acquisition, preferably a 3D ultrasound image. The result of this registration may be applied to subsequently acquired echo or ultrasound images, including two-dimensional ultrasound images, assuming that the ultrasound probe does not move between acquisitions. Thus, in some examples, the registration between the 3D image and the ultrasound image(s) may be performed once for each ultrasound probe position.

At 420, method 400 overlays at least a portion of the planning annotations from the 3D image on the real-time ultrasound image. Since the 3D image and the ultrasound image are co-aligned or registered, the position of particular planning annotations may be ported from the 3D image to the ultrasound image. That is, a planning annotation selectively positioned in the 3D image may be similarly or exactly positioned in the real-time ultrasound image. At 425, method 400 displays the real-time ultrasound image with the overlaid planning annotations, for example via display 134 or display 116. In this way, the operator of the system may view the real-time ultrasound images with pre-operative planning information provided on the display for guidance. It should be appreciated that the operator may selectively toggle one or more of the planning annotations for display. For example, if the planning annotations include indications and delineations of specific anatomical features, but such annotations interfere with the operator's view during the intervention, the operator may select the particular annotation to be removed from the display.

In some examples, the pre-operative planning information may optionally be utilized to augment x-ray images. As an illustrative example, at 430, method 400 controls an x-ray source to generate an x-ray projection of the subject. For example, the method may control an x-ray source such as x-ray tube 104 to generate the x-ray projection of the subject. At 435, method 400 registers the x-ray projection with the ultrasound image or the 3D image. At 440, method 400 overlays the planning annotations from the 3D image on the x-ray projection. At 445, method 400 displays the x-ray projection with the overlaid planning annotations.

It should be appreciated that in some examples, method 400 may not acquire an x-ray projection and therefore may not overlay planning annotations on an x-ray projection. In such examples, method 400 may proceed directly from 425 to 450.

At 450, method 400 determines if the ultrasound probe is moved. If the ultrasound probe is moved (“YES”), method 400 returns to 415. At 415, the method registers the updated real-time ultrasound image with the 3D image, and the method proceeds as described hereinabove. However, if the ultrasound probe is not moved (“NO”), method 400 proceeds to 455. At 455, method 400 ends the ultrasound scan. Method 400 then returns.

A technical effect of the disclosure includes the display of planning annotations over live ultrasound images. Another technical effect of the disclosure includes the display of planning annotations over x-ray projection images. Yet another technical effect of the disclosure includes the registration of live ultrasound images with pre-operative 3D images.

In one embodiment, a method comprises receiving planning annotations of a three-dimensional (3D) image of a subject; during an ultrasound scan of the subject, registering an ultrasound image with the 3D image; overlaying the planning annotations on the ultrasound image; and displaying the ultrasound image with the overlaid planning annotations.

In a first example of the method, the 3D image comprises one of a computed tomography (CT) image or a magnetic resonance imaging (MRI) image, and the ultrasound image comprises a three-dimensional ultrasound image. In a second example of the method optionally including the first example, the method further comprises, responsive to an updated position of an ultrasound probe during the ultrasound scan, registering a second ultrasound image acquired at the updated position with the 3D image, overlaying the planning annotations on the second ultrasound image, and displaying the second ultrasound image with the overlaid planning annotations. In a third example of the method optionally including one or more of the first and second examples, the planning annotations include one or more of an indication of anatomical structures, a delineation of the anatomical structures, spatial measurements, and simulations of device positioning with respect to the anatomical structures. In a fourth example of the method optionally including one or more of the first through third examples, the planning annotations are received from a user via a user interface. In a fifth example of the method optionally including one or more of the first through third examples, the method further comprises removing one or more of the planning annotations from the overlaying of the planning annotations on the ultrasound image responsive to user input. In a sixth example of the method optionally including one or more of the first through fifth examples, only a portion of the planning annotations corresponding to a slice of the 3D image are overlaid on a slice of the ultrasound image. In a seventh example of the method optionally including one or more of the first through sixth examples, the method further comprises, during the ultrasound scan, controlling an x-ray source to generate an x-ray projection of the patient, the x-ray projection comprising a two-dimensional image. In an eighth example of the method optionally including one or more of the first through seventh examples, the method further comprises overlaying the planning annotations on the x-ray projection, and displaying the x-ray projection with the overlaid planning annotations. In a ninth example of the method optionally including one or more of the first through eighth examples, the method further comprises displaying directional information on one or more of the ultrasound image and the x-ray projection.

In another embodiment, a method comprises: acquiring scan data of a subject with an imaging modality; reconstructing a three-dimensional (3D) image from the acquired scan data; receiving annotations for the 3D image; and during an ultrasound scan, overlaying the annotations for the 3D image on an ultrasound image.

In a first example of the method, the method further comprises displaying the ultrasound image with the overlaid annotations. In a second example of the method optionally including the first example, the method further comprises co-aligning the ultrasound image with the 3D image prior to overlaying the annotations on the ultrasound image. In a third example of the method optionally including one or more of the first and second examples, the annotations include one or more of an indication of anatomical structures, a delineation of the anatomical structures, spatial measurements, and simulations of device positioning with respect to the anatomical structures.

In yet another embodiment, a system comprises: a three-dimensional (3D) imaging modality; an ultrasound probe; a user interface; and a processor communicatively coupled to the 3D imaging modality, the ultrasound probe, and the user interface, the processor configured with instructions in non-transitory memory that when executed cause the processor to: acquire, with the 3D imaging modality, a 3D image of a subject; receive, via the user interface, annotations for the 3D image; and during an ultrasound scan of the subject with the ultrasound probe, overlay the annotations for the 3D image on an ultrasound image.

In a first example of the system, the system further comprises a display device communicatively coupled to the processor, wherein the processor is further configured to display the ultrasound image with the overlaid annotations. In a second example of the system optionally including the first example, the processor is further configured to co-align the ultrasound image with the 3D image prior to overlaying the annotations on the ultrasound image. In a third example of the system optionally including one or more of the first and second examples, the annotations include one or more of an indication of anatomical structures, a delineation of the anatomical structures, spatial measurements, and simulations of device positioning with respect to the anatomical structures. In a fourth example of the system optionally including one or more of the first through third examples, the processor is further configured to, responsive to an updated position of an ultrasound probe during the ultrasound scan, register a second ultrasound image acquired at the updated position with the 3D image, overlay the planning annotations on the second ultrasound image, and display the second ultrasound image with the overlaid planning annotations. In a fifth example of the system optionally including one or more of the first through fourth examples, the processor is further configured to remove one or more of the annotations from the overlaying of the annotations on the ultrasound image responsive to user input.

In one representation, a method comprises: receiving planning annotations of a computed tomography (CT) image of a subject; during an ultrasound scan of the subject, registering an ultrasound image with the CT image; overlaying the planning annotations on the ultrasound image; and displaying the ultrasound image with the overlaid planning annotations.

In a first example of the method, the method further comprises, during the ultrasound scan, controlling an x-ray source to generate an x-ray projection of the patient. In a second example of the method optionally including the first example, the method further comprises overlaying the planning annotations on the x-ray projection, and displaying the x-ray projection with the overlaid planning annotations. In a third example of the method optionally including one or more of the first and second examples, the method further comprises displaying directional information on one or more of the ultrasound image and the x-ray projection. In a fourth example of the method optionally including one or more of the first through third examples, the CT image comprises a three-dimensional CT image, the ultrasound image comprises a three-dimensional ultrasound image, and the x-ray projection comprises a two-dimensional x-ray image. In a fifth example of the method optionally including one or more of the first through fourth examples, the method further comprises responsive to an updated position of an ultrasound probe during the ultrasound scan, registering a second ultrasound image acquired at the updated position with the CT image, overlaying the planning annotations on the second ultrasound image, and displaying the second ultrasound image with the overlaid planning annotations. In a sixth example of the method optionally including one or more of the first through fifth examples, the planning annotations include one or more of an indication of anatomical structures, a delineation of the anatomical structures, spatial measurements, and simulations of device positioning with respect to the anatomical structures. In a seventh example of the method optionally including one or more of the first through sixth examples, the planning annotations are received from a user via a user interface. In an eighth example of the method optionally including one or more of the first through seventh examples, the method further comprises removing one or more of the planning annotations from the overlaying of the planning annotations on the ultrasound image responsive to user input. In a ninth example of the method optionally including one or more of the first through eighth examples, only a portion of the planning annotations corresponding to a slice of the CT image are overlaid on a slice of the ultrasound image.

In another representation, a method comprises: acquiring computed tomography (CT) projection data of a subject; reconstructing a CT image from the CT projection data; receiving annotations for the CT image; and during an ultrasound scan, overlaying the annotations for the CT image on an ultrasound image.

In a first example of the method, the method further comprises displaying the ultrasound image with the overlaid annotations. In a second example of the method optionally including the first example, the method further comprises co-aligning the ultrasound image with the CT image prior to overlaying the annotations on the ultrasound image. In a third example of the method optionally including one or more of the first and second examples, the annotations include one or more of an indication of anatomical structures, a delineation of the anatomical structures, spatial measurements, and simulations of device positioning with respect to the anatomical structures.

In yet another representation, a system comprises: a computed tomography (CT) imaging system; an ultrasound probe; a user interface; and a processor communicatively coupled to the CT imaging system, the ultrasound probe, and the user interface, the processor configured with instructions in non-transitory memory that when executed cause the processor to: acquire, with the CT imaging system, projection data of a subject; reconstruct a CT image from the acquired projection data; receive, via the user interface, annotations for the CT image; and during an ultrasound scan of the subject with the ultrasound probe, overlay the annotations for the CT image on an ultrasound image.

In a first example of the system, the system further comprises a display device communicatively coupled to the processor, wherein the processor is further configured to display the ultrasound image with the overlaid annotations. In a second example of the system optionally including the first example, the processor is further configured to co-align the ultrasound image with the CT image prior to overlaying the annotations on the ultrasound image. In a third example of the system optionally including one or more of the first and second examples, the annotations include one or more of an indication of anatomical structures, a delineation of the anatomical structures, spatial measurements, and simulations of device positioning with respect to the anatomical structures. In a fourth example of the system optionally including one or more of the first through third examples, the processor is further configured to, responsive to an updated position of an ultrasound probe during the ultrasound scan, register a second ultrasound image acquired at the updated position with the CT image, overlay the planning annotations on the second ultrasound image, and display the second ultrasound image with the overlaid planning annotations. In a fifth example of the system optionally including one or more of the first through fourth examples, the processor is further configured to remove one or more of the annotations from the overlaying of the annotations on the ultrasound image responsive to user input.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method, comprising:
receiving planning annotations of a three-dimensional (3D) image of a subject;
during an ultrasound scan of the subject, registering an ultrasound image with the 3D image;
overlaying the planning annotations on the ultrasound image; and
displaying the ultrasound image with the overlaid planning annotations.

2. The method of claim 1, wherein the 3D image comprises one of a computed tomography (CT) image or a magnetic resonance imaging (MRI) image, and the ultrasound image comprises a three-dimensional ultrasound image.

3. The method of claim 1, further comprising, responsive to an updated position of an ultrasound probe during the ultrasound scan, registering a second ultrasound image acquired at the updated position with the 3D image, overlaying the planning annotations on the second ultrasound image, and displaying the second ultrasound image with the overlaid planning annotations.

4. The method of claim 1, wherein the planning annotations include one or more of an indication of anatomical structures, a delineation of the anatomical structures, spatial measurements, and simulations of device positioning with respect to the anatomical structures.

5. The method of claim 1, wherein the planning annotations are received from a user via a user interface.

6. The method of claim 1, further comprising removing one or more of the planning annotations from the overlaying of the planning annotations on the ultrasound image responsive to user input.

7. The method of claim 1, wherein only a portion of the planning annotations corresponding to a slice of the 3D image are overlaid on a slice of the ultrasound image.

8. The method of claim 1, further comprising, during the ultrasound scan, controlling an x-ray source to generate an x-ray projection of the patient, the x-ray projection comprising a two-dimensional image.

9. The method of claim 8, further comprising overlaying the planning annotations on the x-ray projection, and displaying the x-ray projection with the overlaid planning annotations.

10. The method of claim 9, further comprising displaying directional information on one or more of the ultrasound image and the x-ray projection.

11. A method, comprising:
acquiring scan data of a subject with an imaging modality;
reconstructing a three-dimensional (3D) image from the acquired scan data;
receiving annotations for the 3D image; and
during an ultrasound scan, overlaying the annotations for the 3D image on an ultrasound image.

12. The method of claim 11, further comprising displaying the ultrasound image with the overlaid annotations.

13. The method of claim 11, further comprising co-aligning the ultrasound image with the 3D image prior to overlaying the annotations on the ultrasound image.

14. The method of claim 11, wherein the annotations include one or more of an indication of anatomical structures, a delineation of the anatomical structures, spatial measurements, and simulations of device positioning with respect to the anatomical structures.

15. A system, comprising:
a three-dimensional (3D) imaging modality;
an ultrasound probe;
a user interface; and
a processor communicatively coupled to the 3D imaging modality, the ultrasound probe, and the user interface, the processor configured with instructions in non-transitory memory that when executed cause the processor to:
acquire, with the 3D imaging modality, a 3D image of a subject;
receive, via the user interface, annotations for the 3D image; and
during an ultrasound scan of the subject with the ultrasound probe, overlay the annotations for the 3D image on an ultrasound image.

16. The system of claim 15, further comprising a display device communicatively coupled to the processor, wherein the processor is further configured to display the ultrasound image with the overlaid annotations.

17. The system of claim 15, wherein the processor is further configured to co-align the ultrasound image with the 3D image prior to overlaying the annotations on the ultrasound image.

18. The system of claim 15, wherein the annotations include one or more of an indication of anatomical structures, a delineation of the anatomical structures, spatial measurements, and simulations of device positioning with respect to the anatomical structures.

19. The system of claim 15, wherein the processor is further configured to, responsive to an updated position of an ultrasound probe during the ultrasound scan, register a second ultrasound image acquired at the updated position with the 3D image, overlay the planning annotations on the second ultrasound image, and display the second ultrasound image with the overlaid planning annotations.

20. The system of claim 15, wherein the processor is further configured to remove one or more of the annotations from the overlaying of the annotations on the ultrasound image responsive to user input.
